(19) United States
(12) Patent Application Publication
(10) Pub. No.: US 2025/0360322 A1
(43) Pub. Date: Nov. 27, 2025

(54) Intraoperative Neural Sensing for Deep Brain Stimulation (DBS)

(71) Applicant: Boston Scientific Neuromodulation Corporation, Valencia, CA (US)

(72) Inventors: Raul Serrano Carmona, Los Angeles, CA (US); Luke Edwards, Valencia, CA (US); G. Karl Steinke, Valencia, CA (US); Eric Nagaoka, Santa Clarita, CA (US)

(21) Appl. No.: 19/201,680

(22) Filed: May 7, 2025

Related U.S. Application Data
(60) Provisional application No. 63/650,305, filed on May 21, 2024.

(51) Int. Cl. A61N 1/372; A61N 1/05; A61N 1/36

(52) U.S. Cl. CPC A61N 1/37241; A61N 1/0534; A61N 1/36125

(57) ABSTRACT

Systems for facilitating electrical stimulation within a patient's brain and of recording electrical activity within a patient's brain during the implantation of electrode leads in the patient's brain are described. The systems include a modified operating room (OR) cable and/or a sensing adapter that provide electrical connections with the electrode lead(s) and also provide electrical connections to off-lead electrodes that may be configured in electrical contact on the patient's body remote from the electrodes of the electrode lead(s). The off-lead electrodes may be reference electrodes, for example.

### CROSS REFERENCE TO RELATED APPLICATIONS

This application is a non-provisional of U.S. Provisional Patent Application Ser. No. 63/650,305, filed May 21, 2024, which is incorporated herein by reference in its entirety, and to which priority is claimed.

### FIELD OF THE INVENTION

This application relates to deep brain stimulation (DBS), and more particularly, to methods and systems for using sensed neural responses for facilitating aspects of DBS.

### Introduction

Implantable neurostimulator devices are devices that generate and deliver electrical stimuli to body nerves and tissues for the therapy of various biological disorders, such as pacemakers to treat cardiac arrhythmia, defibrillators to treat cardiac fibrillation, cochlear stimulators to treat deafness, retinal stimulators to treat blindness, muscle stimulators to produce coordinated limb movement, spinal cord stimulators to treat chronic pain, cortical and deep brain stimulators to treat motor and psychological disorders, and other neural stimulators to treat urinary incontinence, sleep apnea, shoulder subluxation, etc. The description that follows will generally focus on the use of the invention within a Deep Brain Stimulation (DBS) context. DBS has been applied therapeutically for the treatment of neurological disorders, including Parkinson's Disease (PD), essential tremor, dystonia, and epilepsy, to name but a few. Further details discussing the treatment of diseases using DBS are disclosed in U.S. Pat. Nos. 6,845,267, and 6,950,707. However, the present invention may find applicability with any implantable neurostimulator device system.

Each of these neurostimulation systems, whether implantable or external, typically includes one or more electrode-carrying stimulation leads, which are implanted at the desired stimulation site, and a neurostimulator, used externally or implanted remotely from the stimulation site, but coupled either directly to the neurostimulation lead(s) or indirectly to the neurostimulation lead(s) via a lead extension. The neurostimulation system may further comprise a handheld external control device to remotely instruct the neurostimulator to generate electrical stimulation pulses in accordance with selected stimulation parameters. Typically, the stimulation parameters programmed into the neurostimulator can be adjusted by manipulating controls on the external control device to modify the electrical stimulation provided by the neurostimulator system to the patient.

Thus, in accordance with the stimulation parameters programmed by the external control device, electrical pulses can be delivered from the neurostimulator to the stimulation electrode(s) to stimulate or activate a volume of tissue in accordance with a set of stimulation parameters and provide the desired efficacious therapy to the patient. The best stimulus parameter set will typically be one that delivers stimulation energy to the volume of tissue that must be stimulated in order to provide the therapeutic benefit (e.g., treatment of movement disorders), while minimizing the volume of non-target tissue that is stimulated. A typical stimulation parameter set may include the electrodes that are acting as anodes or cathodes, as well as the amplitude, duration, and rate of the stimulation pulses.

Non-optimal electrode placement and stimulation parameter selections may result in excessive energy consumption due to stimulation that is set at too high amplitude, too wide a pulse duration, or too fast a frequency; inadequate or marginalized treatment due to stimulation that is set at too low an amplitude, too narrow a pulse duration, or too slow a frequency; or stimulation of neighboring cell populations that may result in undesirable side effects. For example, bilateral DBS of the subthalamic nucleus has been shown to provide effective therapy for improving the major motor signs of advanced Parkinson's disease, and although the bilateral stimulation of the subthalamic nucleus is considered safe, an emerging concern is the potential negative consequences that it may have on cognitive functioning and overall quality of life (see A. M. M. Frankemolle, et al., Reversing Cognitive-Motor Impairments in Parkinson's Disease Patients Using a Computational Modelling Approach to Deep Brain Stimulation Programming, Brain 2010; pp. 1-16). In large part, this phenomenon is due to the small size of the subthalamic nucleus. Even with the electrodes located predominately within the sensorimotor territory, the electrical field generated by DBS is non-discriminately applied to all neural elements surrounding the electrodes, thereby resulting in the spread of current to neural elements affecting cognition. As a result, diminished cognitive function during stimulation of the subthalamic nucleus may occur do to non-selective activation of non-motor pathways within or around the subthalamic nucleus.

The large number of electrodes available, combined with the ability to generate a variety of complex stimulation pulses, presents a huge selection of stimulation parameter sets to the clinician or patient. In the context of DBS, neurostimulation leads with a complex arrangement of electrodes that not only are distributed axially along the leads, but are also distributed circumferentially around the neurostimulation leads as segmented electrodes, can be used.

To facilitate such selection, the clinician generally programs the external control device, and if applicable the neurostimulator, through a computerized programming system. This programming system can be a self-contained hardware/software system, or can be defined predominantly by software running on a standard personal computer (PC) or mobile platform. The PC or custom hardware may actively control the characteristics of the electrical stimulation generated by the neurostimulator to allow the optimum stimulation parameters to be determined based on patient feedback and to subsequently program the external control device with the optimum stimulation parameters.

When electrical leads are implanted within the patient, the computerized programming system may be used to instruct the neurostimulator to apply electrical stimulation to test placement of the leads and/or electrodes, thereby assuring that the leads and/or electrodes are implanted in effective locations within the patient. The system may also instruct the user how to improve the positioning of the leads, or confirm when a lead is well-positioned. Once the leads are correctly positioned, a fitting procedure, which may be referred to as a navigation session, may be performed using the computerized programming system to program the external control device, and if applicable the neurostimulator, with a set of stimulation parameters that best addresses the neurological disorder(s).

### SUMMARY

Disclosed herein is a system for providing electrical stimulation to a patient's brain using one or more electrode leads implantable in the patient's brain, wherein each of the one or more electrode leads comprise a plurality of distal electrode contacts configured to contact the patient's brain and a plurality of proximal electrode contacts outside the patient's brain, the system comprising: an operating room (OR) cable comprising: a proximal end configured to connect to an external trial stimulator (ETS); a distal end comprising one or more electrode lead connectors configured to connect to the proximal electrode contacts of the one or more electrode leads; and at least two off-lead electrode connectors configured to connect to off-lead electrode contacts that are not configured within the one or more electrode leads. According to some embodiments, at least one of the off-lead electrode connectors is at the proximal end of the OR cable and at least one of the off-lead electrode connectors is at the distal end of the OR cable. According to some embodiments, the OR cable comprises: an ETS connector at the proximal end, wherein the ETS connector is configured to connect to the ETS; and a cable body connected to the ETS connector at its proximal end and connected to the electrode lead connector at its distal end, wherein the cable body comprises a plurality of wires configured within an insulating sheath. According to some embodiments, the ETS comprises: a plurality of stimulation/sensing electrode nodes configured to provide stimulation waveforms and to sense electrical signals; and a plurality of reference electrode nodes configured to provide a voltage reference with respect to electrical signals sensed at the stimulation/sensing nodes. According to some embodiments, the ETS connector comprises: a first plurality of conductors configured to electrically connect to the stimulation/sensing electrode nodes, and a second plurality of conductors configured to electrically connect to the reference electrode nodes. According to some embodiments, the first plurality of conductors is configured to electrically connect the stimulation/sensing electrode nodes of the ETS to a first one or more of the plurality of wires, wherein the first one or more of the plurality of wires each terminate at a lead connector contact within the one or more lead connectors. According to some embodiments, at least one of the second plurality of conductors is configured to electrically connect one or more of the reference electrode nodes to a second one or more of the plurality of wires, wherein the second one or more of the plurality of wires each terminate at an off-lead electrode connector at the distal end of the OR cable. According to some embodiments, the off-lead electrode connector at the distal end of the OR cable comprises a strand of wire that exits the OR cable body proximal to the one or more lead connectors. According to some embodiments, the strand of wire terminates at an alligator clip, a mini-DIN connector, a safety DIN connector, or an EEG DIN connector. According to some embodiments, at least one of the second plurality of conductors is configured to electrically connect one or more of the reference electrode nodes of the ETS to one or more off-lead electrode connectors configured as part of the ETS connector. According to some embodiments, each of the one or more off-lead electrode connectors configured as part of the ETS connector comprises a jack configured to attach to a proximal end of an off-lead electrode cable that is configured to attach to a off-lead electrode at the distal end of the off-lead electrode cable. According to some embodiments, the off-lead electrode is a patch electrode. According to some embodiments, the ETS connector comprises two or more off-lead electrode connectors. According to some embodiments, the system further comprises the ETS. According to some embodiments, the off-lead electrode is a reference electrode.

Also disclosed herein is a system for providing electrical stimulation to a patient's brain using one or more electrode leads implantable in the patient's brain, wherein each of the one or more electrode leads comprise a plurality of distal electrode contacts configured to contact the patient's brain and a plurality of proximal electrode contacts outside the patient's brain, the system comprising: a sensing adapter comprising: an input port configured to connect to an external trial stimulator (ETS); one or more output ports each configured to connect to a proximal end of an operating room (OR) cable, wherein the OR cable comprises a distal end configured to connect to the proximal electrode contacts of the one or more electrode leads; and at least two off-lead electrode connectors each configured to connect to a proximal end of an off-lead electrode cable. According to some embodiments, the ETS comprises: a plurality of stimulation/sensing electrode nodes configured to provide stimulation waveforms and to sense electrical signals; and a plurality of reference electrode nodes configured to provide a voltage reference with respect to electrical signals sensed at the stimulation/sensing nodes. According to some embodiments, the sensing adapter comprises: a first plurality of conductors configured to electrically connect the stimulation/sensing electrode nodes to the one or more output ports, and a second plurality of conductors each configured to electrically connect the reference electrode nodes to the off-lead electrode connectors. According to some embodiments, the system further comprises one or more off-lead electrode cables each having a proximal end configured to connect to the off-lead electrode connectors of the sensing adapter. According to some embodiments, each of the off-lead electrode cables comprise a distal end configured to connect to a off-lead electrode. According to some embodiments, the off-lead electrode comprises a patch electrode. According to some embodiments, the distal end of the off-lead electrode cable comprises an alligator clip, a mini-DIN connector, a safety DIN connector, or an EEG DIN connector. According to some embodiments, the system further comprises the ETS. According to some embodiments, the off-lead electrode is a reference electrode.

Also disclosed herein is a method for providing electrical stimulation to a patient's brain using one or more electrode leads implantable in the patient's brain, wherein each of the one or more electrode leads comprise a plurality of distal electrode contacts configured to contact the patient's brain and a plurality of proximal electrode contacts outside the patient's brain, the method comprising: providing an operating room (OR) cable comprising: a proximal end configured to connect to an external trial stimulator (ETS), a distal end comprising one or more electrode lead connectors configured to connect to the proximal electrode contacts of the one or more electrode leads, and at least two off-lead electrode connectors configured to connect to off-lead electrode contacts that are not configured within the one or more electrode leads. The method comprises connecting the proximal end of the OR cable to the ETS, connecting the one or more electrode lead connectors to the proximal electrode contacts of the one or more electrode leads, connecting a first of the at least one of the off-lead electrode connectors to a first off-lead electrode, configuring the first off-lead electrode to have electrical continuity with the patient, and using the ETS to provide one or more stimulation waveforms to one or more of the plurality of proximal electrode contacts of the one or more electrode leads. According to some embodiments, the first off-lead electrode is a patch electrode and wherein configuring the first off-lead electrode to have electrical continuity with the patient comprises attaching the patch electrode to the patient's skin. According to some embodiments, attaching the patch electrode to the patient's skin comprises attaching the patch electrode outside a sterile field of the patient. According to some embodiments, attaching the patch electrode to the patient's skin comprises attaching the patch electrode inside a sterile field of the patient. According to some embodiments, the first off-lead electrode is a clip, and wherein configuring the first off-lead electrode to have electrical continuity with the patient comprises attaching the clip to a cannula inserted into the patient's brain. According to some embodiments, the first off-lead electrode is a reference electrode.

Also disclosed herein is a method for providing electrical stimulation to a patient's brain using one or more electrode leads implantable in the patient's brain, wherein each of the one or more electrode leads comprise a plurality of distal electrode contacts configured to contact the patient's brain and a plurality of proximal electrode contacts outside the patient's brain, the method comprising: providing a sensing adapter comprising: an input port configured to connect to an external trial stimulator (ETS); one or more output ports each configured to connect to a proximal end of an operating room (OR) cable, wherein the OR cable comprises a distal end configured to connect to the proximal electrode contacts of the one or more electrode leads; and at least two off-lead electrode connectors each configured to connect to a proximal end of an off-lead electrode cable. The method further comprises connecting the input port to the ETS, connecting the one or more output ports to the proximal end of the OR cable connecting the distal end of the OR cable to the proximal electrode contacts of the one or more electrode leads, connecting a first of the at least one of the off-lead electrode connectors to a first off-lead electrode cable, connecting a distal end of the first off-lead electrode cable to a first off-lead electrode, configuring the first off-lead electrode to have electrical continuity with the patient, and using the ETS to provide one or more stimulation waveforms to one or more of the plurality of proximal electrode contacts of the one or more electrode leads. According to some embodiments, the first off-lead electrode is a patch electrode and wherein configuring the first off-lead electrode to have electrical continuity with the patient comprises attaching the patch electrode to the patient's skin. According to some embodiments, attaching the patch electrode to the patient's skin comprises attaching the patch electrode outside a sterile field of the patient. According to some embodiments, attaching the patch electrode to the patient's skin comprises attaching the patch electrode inside a sterile field of the patient. According to some embodiments, the first off-lead electrode is a clip, and wherein configuring the first off-lead electrode to have electrical continuity with the patient comprises attaching the clip to a cannula inserted into the patient's brain. According to some embodiments, the first off-lead electrode is a reference electrode.

The invention may also reside in the form of a programed external device (via its control circuitry) for carrying out the above methods, a programmed implantable pulse generator (IPG) or external trial stimulator (ETS) (via its control circuitry) for carrying out the above methods, a system including a programmed external device and IPG or ETS for carrying out the above methods, or as a computer-readable media for carrying out the above methods stored in an external device or IPG or ETS. The invention may also reside in one or more non-transitory computer-readable media comprising instructions, which when executed by a processor of a machine configure the machine to perform any of the above methods.

### BRIEF DESCRIPTION OF THE DRAWINGS

FIG. 1A shows an Implantable Pulse Generator (IPG).

FIG. 1B shows a percutaneous lead having split-ring electrodes.

FIGS. 2A and 2B show an example of stimulation pulses (waveforms) producible by the IPG or by an External Trial Stimulator (ETS).

FIG. 3 shows an example of stimulation circuitry useable in the IPG or ETS.

FIG. 4 shows an ETS environment useable to provide stimulation before implantation of an IPG.

FIG. 5 shows various external devices capable of communicating with and programming stimulation in an IPG or ETS.

FIG. 6 illustrates sensing circuitry useable in an IPG.

FIG. 7 illustrates an embodiment of a user interface (UI) for programming stimulation.

FIG. 8 illustrates evoked resonant neural activity (ERNA) and a motor evoked potential (MEP).

FIG. 9 illustrates an operating room system comprising an ETS for providing stimulation and recording electrical signals in a patient's brain.

FIG. 10 illustrates a system comprising an embodiment of an operating room (OR) cable as described herein.

FIG. 11 illustrates a system comprising a sensing adapter as described herein.

FIG. 12 shows a configuration using an embodiment of an OR cable as described herein.

FIG. 13 shows a configuration using an embodiment of an OR cable as described herein.

FIG. 14 shows a configuration using an embodiment of a sensing adapter as described herein.

### DETAILED DESCRIPTION

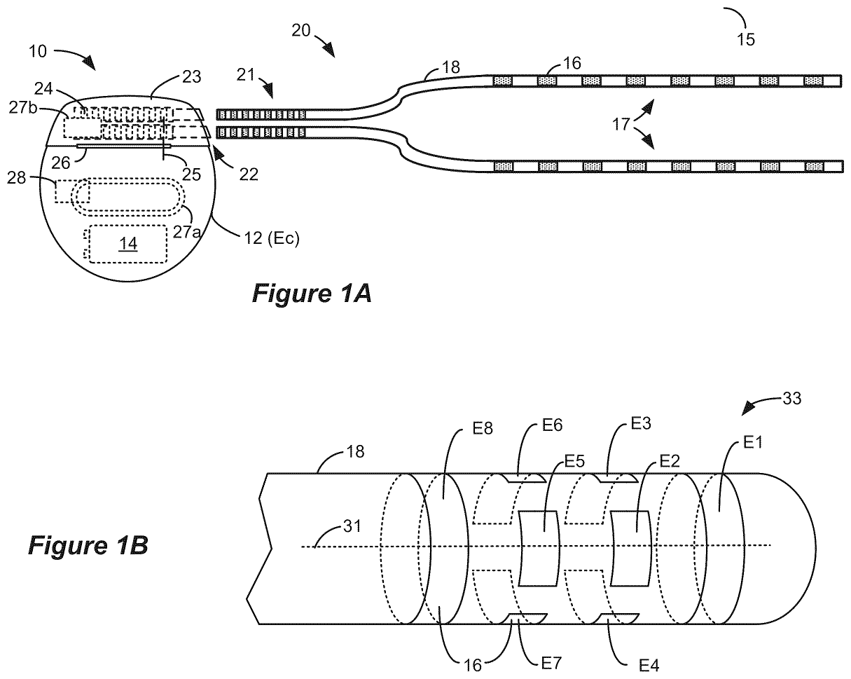

An implantable stimulator system typically includes an Implantable Pulse Generator (IPG) 10 shown in FIG. 1A. The IPG 10 includes a biocompatible device case 12 that holds the circuitry and a battery 14 for providing power for the IPG to function. The IPG 10 is coupled to tissue-stimulating electrode contacts 16 via one or more electrode leads 18 that form an electrode array 17. In another example shown in FIG. 1B, an electrode array 33 can include one or more split-ring electrode contacts. In this example, eight electrode contacts 16 (E1-E8) are shown on the electrode lead 18. Electrode contact E1 at the distal end of the lead and electrode contact E8 at a proximal end of the lead comprise ring electrode contacts spanning 360 degrees around a central axis of the lead 18. In some embodiments, the electrode contact E1 may be a “bullet tip” electrode contact, meaning that it can cover the tip of the electrode lead. Electrode contacts E2, E3, and E4 comprise split-ring electrode contacts, each of which are located at the same longitudinal position along the central axis 31, but with each spanning less than 360 degrees around the axis. For example, each of the electrode contacts E2, E3, and E4 may span 90 degrees around the axis 31, with each being separated from the others by gaps of 30 degrees. Electrode contacts E5, E6, and E7 also comprise split-ring electrodes, but are located at a different longitudinal position along the central axis 31 than are split ring electrode contacts E4, E2, and E3. As shown, the split-ring electrode contacts E2-E4 and E5-E7 may be located at longitudinal positions along the axis 31 between ring electrode contacts E1 and E8. However, this is just one example of a lead 18 having split-ring electrode contacts. In other designs, all electrode contacts can be split-ring, or there could be different numbers of split-ring electrode contacts at each longitudinal position (i.e., more or less than three), or the ring and split-ring electrode contacts could occur at different or random longitudinal positions, etc.

Referring again to FIG. 1A, lead wires (not shown) within the electrode leads 18 are coupled to the electrode contacts 16 and to proximal contacts 21 that are insertable into lead connectors 22 fixed in a header 23 on the IPG 10, which header can comprise an epoxy for example. Once inserted, the proximal contacts 21 connect to header contacts 24 within the lead connectors 22, which are in turn coupled by feedthrough pins 25 through a case feedthrough 26 to stimulation circuitry 28 within the case 12, which stimulation circuitry 28 is described below.

In the IPG 10 illustrated in FIG. 1A, there are sixteen electrode contacts, split between two percutaneous leads 18, and thus the header 23 may include two eight electrode lead connectors 22. However, the type and number of leads, and the number of electrodes, in an IPG is application-specific and therefore can vary. For example, the IPG 10 may include a 2×2 array of eight-electrode lead connectors, wherein two of the lead connectors would not be used in the illustrated embodiment. The conductive case 12 can also comprise an electrode (Ec).

In a DBS application, as is useful in the treatment of tremor in Parkinson's disease for example, the IPG 10 is typically implanted under the patient's clavicle (collarbone). The electrode leads 18 are tunneled through the neck and the scalp and the electrode leads 18 are implanted through holes drilled in the skull and positioned for example in the subthalamic nucleus (STN) and the pedunculopontine nucleus (PPN) in each brain hemisphere. According to some embodiments, lead extensions (not shown) may be used to extend the reach of the electrode leads 18 to connect to the implanted IPG.

IPG 10 can include an antenna 27a allowing it to communicate bi-directionally with a number of external devices discussed subsequently. Antenna 27a as shown comprises a conductive coil within the case 12, although the coil antenna 27a can also appear in the header 23. When antenna 27a is configured as a coil, communication with external devices preferably occurs using near-field magnetic induction. IPG 10 may also include a Radio-Frequency (RF) antenna 27b. In FIG. 1A, RF antenna 27b is shown within the header 23, but it may also be within the case 12. RF antenna 27b may comprise a patch, slot, or wire, and may operate as a monopole or dipole. RF antenna 27b preferably communicates using far-field electromagnetic waves, and may operate in accordance with any number of known RF communication standards, such as Bluetooth, Bluetooth Low Energy (BLE), as described in U.S. Patent Publication 2019/0209851, Zigbee, WiFi, MICS, and the like.

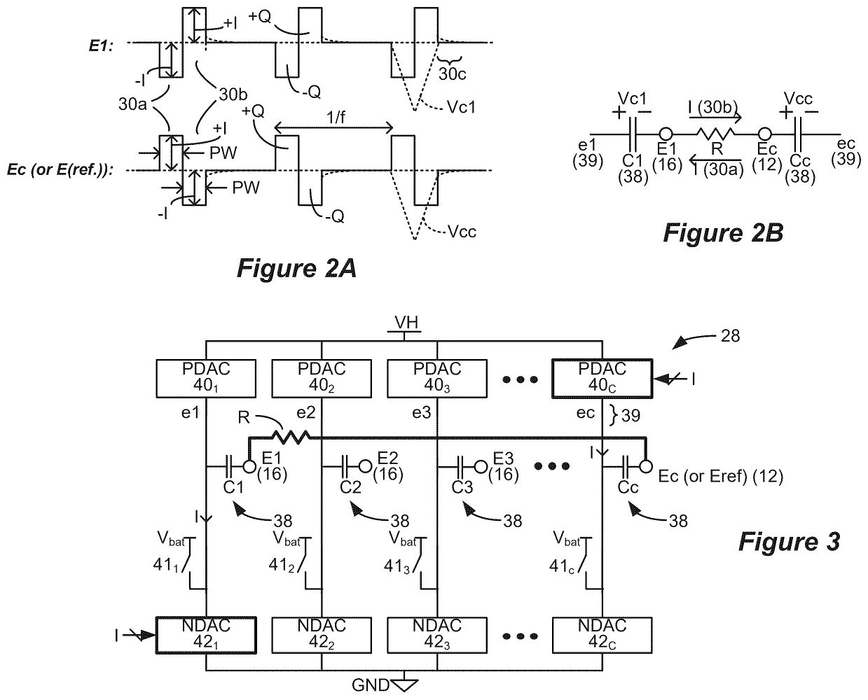

Stimulation in IPG 10 is typically provided by pulses, each of which may include a number of phases such as 30a and 30b, as shown in the example of FIG. 2A. In the example shown, such stimulation is monopolar, meaning that a current is provided between at least one selected lead-based electrode contact (e.g., E1) and the case electrode contact Ec 12. As used herein, the electrode contact Ec 12 may also be referred to as a reference electrode contact Eref, for reasons that will become apparent below. Stimulation parameters typically include amplitude (current I, although a voltage amplitude V can also be used); frequency (f); pulse width (PW) of the pulses or of its individual phases such as 30a and 30b; the electrode contacts 16 selected to provide the stimulation; and the polarity of such selected electrode contacts, i.e., whether they act as anodes that source current to the tissue or cathodes that sink current from the tissue. These and possibly other stimulation parameters taken together comprise a stimulation program that the stimulation circuitry 28 in the IPG 10 can execute to provide therapeutic stimulation to a patient.

In the example of FIG. 2A, electrode contact E1 has been selected as a cathode (during its first phase 30a), and thus provides pulses which sink a negative current of amplitude −I from the tissue. The case electrode contact Ec has been selected as an anode (again during first phase 30a), and thus provides pulses which source a corresponding positive current of amplitude +I to the tissue. Note that at any time the current sunk from the tissue (e.g., −I at E1 during phase 30a) equals the current sourced to the tissue (e.g., +I at Ec during phase 30a) to ensure that the net current injected into the tissue is zero. The polarity of the currents at these electrode contacts can be changed: Ec can be selected as a cathode, and E1 can be selected as an anode, etc.

IPG 10 includes stimulation circuitry 28 to form prescribed stimulation at a patient's tissue. FIG. 3 shows an example of stimulation circuitry 28, which includes one or more current sources 40; and one or more current sinks 42ᵢ. The sources and sinks 40; and 42; can comprise Digital-to-Analog converters (DACs), and may be referred to as PDACs 40; and NDACs 42; in accordance with the Positive (sourced, anodic) and Negative (sunk, cathodic) currents they respectively issue. In the example shown, a NDAC/PDAC 40ᵢ/42ᵢ pair is dedicated (hardwired) to a particular electrode node ei 39. Each electrode node Ei 39 is connected to an electrode contact Ei 16 via a DC-blocking capacitor Ci 38, for the reasons explained below. PDACs 40; and NDACs 42ᵢ can also comprise voltage sources.

Proper control of the PDACs 40; and NDACs 42ᵢ allows any of the electrode contacts 16 and the case/reference electrode Ec (or Eref) 12 to act as anodes or cathodes to create a current through a patient's tissue, R, hopefully with good therapeutic effect. In the example shown, and consistent with the first pulse phase 30a of FIG. 2A, electrode contact E1 has been selected as a cathode electrode to sink current from the tissue R and case electrode Ec has been selected as an anode electrode to source current to the tissue R. Thus, PDAC 40_{C} and NDAC 42₁ are activated and digitally programmed to produce the desired current, I, with the correct timing (e.g., in accordance with the prescribed frequency F and pulse width PW). Power for the stimulation circuitry 28 is provided by a compliance voltage VH, as described in further detail in U.S. Patent Application Publication 2013/0289665.

Other stimulation circuitries 28 can also be used in the IPG 10. In an example not shown, a switching matrix can intervene between the one or more PDACs 40; and the electrode nodes ei 39, and between the one or more NDACs 42; and the electrode nodes. Switching matrices allows one or more of the PDACs or one or more of the NDACs to be connected to one or more electrode nodes at a given time. Various examples of stimulation circuitries can be found in U.S. Pat. Nos. 6,181,969, 8,606,362, 8,620,436, U.S. Patent Application Publications 2018/0071520 and 2019/0083796. The stimulation circuitries described herein provide multiple independent current control (MICC) (or multiple independent voltage control) to guide the estimate of current fractionalization among multiple electrodes and estimate a total amplitude that provide a desired strength. In other words, the total anodic (or cathodic) current can be split among two or more electrodes and/or the total cathodic current can be split among two or more electrodes, allowing the stimulation location and resulting field shapes to be adjusted. For example, a “virtual electrode” may be created at a position between two physical electrode contacts by fractionating current between the two electrodes.

Much of the stimulation circuitry 28 of FIG. 3, including the PDACs 40ᵢ and NDACs 42ᵢ, the switch matrices (if present), and the electrode nodes ei 39 can be integrated on one or more Application Specific Integrated Circuits (ASICs), as described in U.S. Patent Application Publications 2012/0095529, 2012/0092031, and 2012/0095519. As explained in these references, ASIC(s) may also contain other circuitry useful in the IPG 10, such as telemetry circuitry (for interfacing off chip with telemetry antennas 27a and/or 27b), circuitry for generating the compliance voltage VH, various measurement circuits, etc.

Also shown in FIG. 3 are DC-blocking capacitors Ci 38 placed in series in the electrode current paths between each of the electrode nodes ei 39 and the electrodes Ei 16 (including the case electrode Ec 12). The DC-blocking capacitors 38 act as a safety measure to prevent DC current injection into the patient, as could occur for example if there is a circuit fault in the stimulation circuitry 28. The DC-blocking capacitors 38 are typically provided off-chip (off of the ASIC(s)), and instead may be provided in or on a circuit board in the IPG 10 used to integrate its various components, as explained in U.S. Patent Application Publication 2015/0157861.

Referring again to FIG. 2A, the stimulation pulses as shown are biphasic, with each pulse comprising a first phase 30a followed thereafter by a second phase 30b of opposite polarity. Biphasic pulses are useful to actively recover any charge that might be stored on capacitive elements in the electrode current paths, such as on the DC-blocking capacitors 38. Charge recovery is shown with reference to both FIGS. 2A and 2B. During the first pulse phase 30a, charge will build up across the DC-blocking capacitors C1 and Cc associated with the electrodes E1 and Ec used to produce the current, giving rise to voltages Vc1 and Vcc which decrease in accordance with the amplitude of the current and the capacitance of the capacitors 38 (dV/dt=I/C). During the second pulse phase 30b, when the polarity of the current I is reversed at the selected electrodes E1 and Ec, the stored charge on capacitors C1 and Cc is actively recovered, and thus voltages Vc1 and Vcc increase and return to 0V at the end of the second pulse phase 30b.

To recover all charge by the end of the second pulse phase 30b of each pulse (Vc1=Vcc =0V), the first and second phases 30a and 30b are charged balanced at each electrode, with the first pulse phase 30a providing a charge of −Q(−I*PW) and the second pulse phase 30b providing a charge of +Q(+I*PW) at electrode E1, and with the first pulse phase 30a providing a charge of +Q and the second pulse phase 30b providing a charge of −Q at the case electrode Ec. In the example shown, such charge balancing is achieved by using the same pulse width (PW) and the same amplitude ([I]) for each of the opposite-polarity pulse phases 30a and 30b. However, the pulse phases 30a and 30b may also be charged balanced at each electrode if the product of the amplitude and pulse widths of the two phases 30a and 30b are equal, or if the area under each of the phases is equal, as is known.

FIG. 3 shows that stimulation circuitry 28 can include passive recovery switches 41i, which are described further in U.S. Patent Application Publications 2018/0071527 and 2018/0140831. Passive recovery switches 41; may be attached to each of the electrode nodes ei 39, and are used to passively recover any charge remaining on the DC-blocking capacitors Ci 38 after issuance of the second pulse phase 30b—i.e., to recover charge without actively driving a current using the DAC circuitry. Passive charge recovery can be prudent, because non-idealities in the stimulation circuitry 28 may lead to pulse phases 30a and 30b that are not perfectly charge balanced.

Therefore, and as shown in FIG. 2A, passive charge recovery typically occurs after the issuance of second pulse phases 30b, for example during at least a portion 30c of the quiet periods between the pulses, by closing passive recovery switches 41ᵢ. As shown in FIG. 3, the other end of the switches 41ᵢ not coupled to the electrode nodes ei 39 are connected to a common reference voltage, which in this example comprises the voltage of the battery 14, Vbat, although another reference voltage could be used. As explained in the above-cited references, passive charge recovery tends to equilibrate the charge on the DC-blocking capacitors 38 by placing the capacitors in parallel between the reference voltage (Vbat) and the patient's tissue. Note that passive charge recovery is illustrated as small exponentially decaying curves during 30c in FIG. 2A, which may be positive or negative depending on whether pulse phase 30a or 30b have a predominance of charge at a given electrode.

Passive charge recovery 30c may alleviate the need to use biphasic pulses for charge recovery, especially in the DBS context when the amplitudes of currents may be lower, and therefore charge recovery is less of a concern. For example, and although not shown in FIG. 2A, the pulses provided to the tissue may be monophasic, comprising only a first pulse phase 30a. This may be followed thereafter by passive charge recovery 30c to eliminate any charge build up that occurred during the singular pulses 30a.

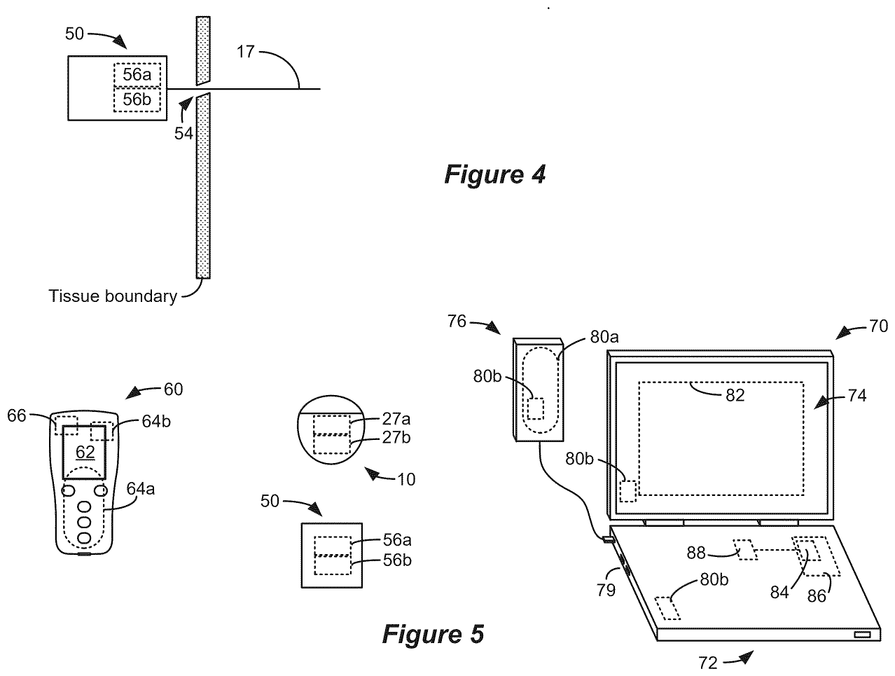

FIG. 4 shows an external trial stimulation environment that may precede implantation of an IPG 10 in a patient, for example, during the operating room to test stimulation and confirm the lead position. During external trial stimulation, stimulation can be tried on the implant patient to evaluate side-effect thresholds and confirm that the lead is not too close to structures that cause side effects. During the external trial stimulation/operating room environment, an IPG has not yet been implanted in the patient. Thus, an external trial stimulator (ETS) 50 is used instead of an IPG. The ETS 50 may include some, or all of the capabilities of an IPG, and may have similar circuitry as an IPG. As used in this disclosure, the ETS 50 may also be referred to as an external pulse generator (EPG), operating room (OR) stimulator, OR box, or the like. As used in this disclosure, the term “ETS” includes all of these modalities and generally refers to any external device that allows a clinician to provide electrical signals to and receive electrical signals from electrodes implanted within the patient and/or electrodes otherwise associated with a patient (such as reference/off-lead electrodes, as discussed below). Aspects of this disclosure are particularly relevant to the operating room environment, and thus aspects of embodiments of an ETS 50 are discussed in more detail below. Like the IPG 10, the external trial stimulator (ETS) 50 can include one or more antennas to enable bi-directional communications with external devices such as those shown in FIG. 5. Such antennas can include a near-field magnetic-induction coil antenna 56a, and/or a far-field RF antenna 56b, as described earlier. ETS 50 may also include stimulation circuitry able to form stimulation in accordance with a stimulation program, which circuitry may be similar to or comprise the same stimulation circuitry 28 (FIG. 3) present in the IPG 10. ETS 50 may also include a battery (not shown) for operational power. The sensing capabilities described herein with regard to the IPG 10, may also be included in the ETS 50 for the purposes described below. As the IPG may include a case electrode, an ETS may provide one or more connections to establish similar returns; for example, using patch electrodes. Likewise, the ETS may communicate with the clinician programmer (CP) so that the CP can process the data as described below.

FIG. 5 shows various external devices that can wirelessly communicate data with the IPG 10 or ETS 50, including a patient hand-held external controller 60, and a clinician programmer (CP) 70. Both of devices 60 and 70 can be used to wirelessly transmit a stimulation program to the IPG 10 or ETS 50—that is, to program their stimulation circuitries to produce stimulation with a desired amplitude and timing described earlier. Both devices 60 and 70 may also be used to adjust one or more stimulation parameters of a stimulation program that the IPG 10 is currently executing. Devices 60 and 70 may also wirelessly receive information from the IPG 10 or ETS 50, such as various status information, etc.

A patient's external controller 60 can be as described in U.S. Patent Application Publication 2015/0080982 for example and may comprise a controller dedicated to work with the IPG 10 or ETS 50. External controller 60 may also comprise a general-purpose mobile electronics device such as a mobile phone which has been programmed with a Medical Device Application (MDA) allowing it to work as a wireless controller for the IPG 10 or ETS, as described in U.S. Patent Application Publication 2015/0231402. External controller 60 includes a user interface, preferably including means for entering commands (e.g., buttons or selectable graphical elements) and a display 62. The external controller 60's user interface enables a patient to adjust stimulation parameters, although it may have limited functionality when compared to the more-powerful clinician programmer 70, described shortly.

The external controller 60 can have one or more antennas capable of communicating with the IPG 10. For example, the external controller 60 can have a near-field magnetic-induction coil antenna 64a capable of wirelessly communicating with the coil antenna 27a or 56a in the IPG 10 or ETS 50. The external controller 60 can also have a far-field RF antenna 64b capable of wirelessly communicating with the RF antenna 27b or 56b in the IPG 10 or ETS 50.

Clinician programmer 70 is described further in U.S. Patent Application Publication 2015/0360038, and can comprise a computing device 72, such as a desktop, laptop, or notebook computer, a tablet, a mobile smart phone, a Personal Data Assistant (PDA)-type mobile computing device, etc. In FIG. 5, computing device 72 is shown as a laptop computer that includes typical computer user interface means such as a screen 74, a mouse, a keyboard, speakers, a stylus, a printer, etc., not all of which are shown for convenience. Also shown in FIG. 5 are accessory devices for the clinician programmer 70 that are usually specific to its operation as a stimulation controller, such as a communication “wand” 76 coupleable to suitable ports on the computing device 72, such as USB ports 79 for example.

The antenna used in the clinician programmer 70 to communicate with the IPG 10 or ETS 50 can depend on the type of antennas included in those devices. If the patient's IPG 10 or ETS 50 includes a coil antenna 27a or 56a, wand 76 can likewise include a coil antenna 80a to establish near-field magnetic-induction communications at small distances. In this instance, the wand 76 may be affixed in close proximity to the patient, such as by placing the wand 76 in a belt or holster wearable by the patient and proximate to the patient's IPG 10 or ETS 50. If the IPG 10 or ETS 50 includes an RF antenna 27b or 56b, the wand 76, the computing device 72, or both, can likewise include an RF antenna 80b to establish communication at larger distances. The clinician programmer 70 can also communicate with other devices and networks, such as the Internet, either wirelessly or via a wired link provided at an Ethernet or network port.

To program stimulation programs or parameters for the IPG 10 or ETS 50, the clinician interfaces with a clinician programmer graphical user interface (GUI) 82 provided on the display 74 of the computing device 72. As one skilled in the art understands, the GUI 82 can be rendered by execution of clinician programmer software 84 stored in the computing device 72, which software may be stored in the device's non-volatile memory 86. Execution of the clinician programmer software 84 in the computing device 72 can be facilitated by control circuitry 88 such as one or more microprocessors, microcomputers, FPGAs, DSPs, other digital logic structures, etc., which are capable of executing programs in a computing device, and which may comprise their own memories. For example, control circuitry 88 can comprise an i5 processor manufactured by Intel Corp, as described at https://www.intel.com/content/www/us/en/products/processors/core/i5-processors.html. Such control circuitry 88, in addition to executing the clinician programmer software 84 and rendering the GUI 82, can also enable communications via antennas 80a or 80b to communicate stimulation parameters chosen through the GUI 82 to the patient's IPG 10.

The user interface of the external controller 60 may provide similar functionality because the external controller 60 can include similar hardware and software programming as the clinician programmer. For example, the external controller 60 includes control circuitry 66 similar to the control circuitry 88 in the clinician programmer 70 and may similarly be programmed with external controller software stored in device memory.

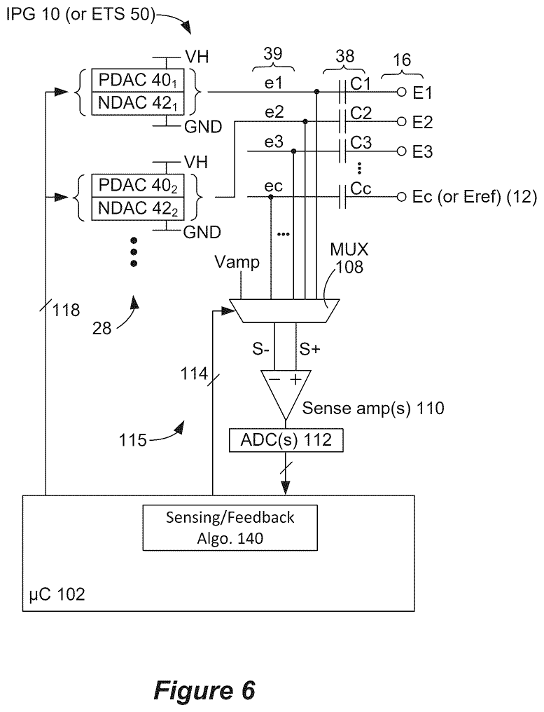

Aspects of this disclosure relate to stimulation systems that include sensing capability to complement the stimulation that such systems provide. FIG. 6 shows an embodiment of circuitry that includes both stimulation and sensing functionality. The illustrated circuitry may be comprised within an IPG 10 or within an ETS 50. As mentioned above, this disclosure is primarily focused on the operating room and/or clinical environment, so most of the embodiments discussed herein relate to circuitry comprised within an ETS 50. Since, in such an environment, the IPG (and its metallic case) has not yet been implanted in the patient, the electrode Ec/Eref 12 is primarily referred to as a reference electrode Eref.

The illustrated circuitry may provide stimulation and sensing innate or evoked signals. The ETS 50 includes control circuitry 102, which may comprise a microcontroller, such as Part Number MSP430, manufactured by Texas Instruments, Inc., which is described in data sheets at http://www.ti.com/microcontrollers/msp430-ultra-low-power-mcus/overview.html, which are incorporated herein by reference. Other types of controller circuitry may be used in lieu of a microcontroller as well, such as microprocessors, FPGAs, DSPs, or combinations of these, etc. Control circuitry 102 may also be formed in whole or in part in one or more Application Specific Integrated Circuits (ASICs), such as those described and incorporated earlier. The control circuitry 102 may be configured with one or more sensing/feedback algorithms 140 that are configured to cause the IPG/ETS to sense neural signals and make certain adjustments and/or take certain actions based on the sensed neural signals.

The ETS 50 also includes stimulation circuitry 28 to produce stimulation at the electrodes 16, which may comprise the stimulation circuitry 28 shown earlier (FIG. 3). A bus 118 provides digital control signals from the control circuitry 102 to one or more PDACs 40; or NDACs 42; to produce currents or voltages of prescribed amplitudes (I) for the stimulation pulses, and with the correct timing (PW, F) at selected electrodes. As noted earlier, the DACs can be powered between a compliance voltage VH and ground. As also noted earlier, but not shown in FIG. 4, switch matrices could intervene between the PDACs and the electrode nodes 39, and between the NDACs and the electrode nodes 39, to route their outputs to one or more of the electrodes, including the reference electrode 12 (Eref), as described in more detail below. Control signals for switch matrices, if present, may also be carried by bus 118. Notice that the current paths to the electrodes 16 include the DC-blocking capacitors 38 described earlier, which provide safety by preventing the inadvertent supply of DC current to an electrode and to a patient's tissue. Passive recovery switches 41; (FIG. 3) could also be present but are not shown in FIG. 6 for simplicity.

ETS 50 also includes sensing circuitry 115, and one or more of the electrodes 16 can be used to sense innate or evoked electrical signals, e.g., biopotentials from the patient's tissue. In this regard, each electrode node 39 can further be coupled to a sense amp circuit 110. Under control by bus 114, a multiplexer 108 can select one or more electrodes to operate as sensing electrodes (S+, S−) by coupling the electrode(s) to the sense amps circuit 110 at a given time, as explained further below. Although only one multiplexer 108 and sense amp circuit 110 are shown in FIG. 6, there could be more than one. For example, there can be four multiplexer 108/sense amp circuit 110 pairs each operable within one of four timing channels supported by the IPG 10 to provide stimulation. The sensed signals output by the sense amp circuitry are preferably converted to digital signals by one or more Analog-to-Digital converters (ADC(s)) 112, which may sample the output of the sense amp circuit 110 at 50 kHz for example. The ADC(s) 112 may also reside within the control circuitry 102, particularly if the control circuitry 102 has A/D inputs. Multiplexer 108 can also provide a fixed reference voltage, Vamp, to the sense amp circuit 110, as is useful in a single-ended sensing mode (i.e., to set S− to Vamp). Further aspects of sensing circuitry, and particularly, the various configuration of sense amps may be found in U.S. Pat. No. 11,633,138, issued Apr. 25, 2023, and U.S. Patent Application Publication No. 2023/0173273, published Jun. 8, 2023, both of which are incorporated herein by reference.

So as not to bypass the safety provided by the DC-blocking capacitors 38, the inputs to the sense amp circuitry 110 are preferably taken from the electrode nodes 39. However, the DC-blocking capacitors 38 will pass AC signal components (while blocking DC components), and thus AC components within the signals being sensed will still readily be sensed by the sense amp circuitry 110. In other examples, signals may be sensed directly at the electrodes 16 without passage through intervening capacitors 38.

According to some embodiments, it may be preferred to sense signals differentially, and in this regard, the sense amp circuitry 110 comprises a differential amplifier receiving the sensed signal S+ (e.g., E3) at its non-inverting input and the sensing reference S− (e.g., E1) at its inverting input. As one skilled in the art understands, the differential amplifier will subtract S− from S+ at its output, and so will cancel out any common mode voltage from both inputs. This can be useful for example when sensing various neural signals, as it may be useful to subtract the relatively large-scale stimulation artifact from the measurement (as much as possible). Some embodiments involve sensing signals using an electrode configured on the implanted electrode lead with respect to an externally located (i.e., not located within the patient's brain) reference electrode connected to the electrode node Eref 12, as described in more detail below. Aspects of this discussion will become clearer in view of further discussion below.

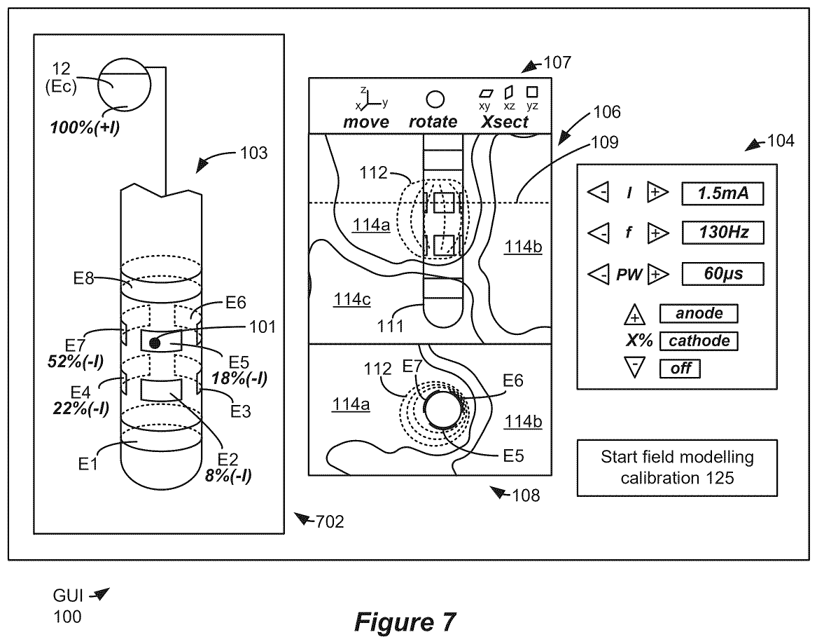

Particularly in the DBS context, it can be useful to provide a clinician with a visual indication of how stimulation selected for a patient will interact with the tissue in which the electrodes are implanted. This is illustrated in FIG. 7, which shows a Graphical User Interface (GUI) 100 operable on an external device capable of communicating with an IPG 10 or ETS 50. Typically, and as assumed in the description that follows, GUI 100 would be rendered on a clinician programmer 70 (FIG. 5), which may be used during surgical implantation of the leads, or after implantation when a therapeutically useful stimulation program is being chosen for a patient. However, GUI 100 could be rendered on a patient external programmer 60 (FIG. 5) or any other external device capable of communicating with the IPG 10 or ETS 50.

GUI 100 allows a clinician (or patient) to select the stimulation program that the IPG 110 or ETS 50 will provide and provides options that control sensing of innate or evoked responses, as described below. In this regard, the GUI 100 may include a stimulation parameter interface 104 where various aspects of the stimulation program can be selected or adjusted. For example, interface 104 allows a user to select the amplitude (e.g., a current I) for stimulation; the frequency (f) of stimulation pulses; and the pulse width (PW) of the stimulation pulses. Stimulation parameter interface 104 can be significantly more complicated, particularly if the IPG 10 or ETS 50 supports the provision of stimulation that is more complicated than a repeating sequence of pulses. See, e.g., U.S. Patent Application Publication 2018/0071513. Nonetheless, interface 104 is simply shown for simplicity in FIG. 7 as allowing only for amplitude, frequency, and pulse width adjustment. Stimulation parameter interface 104 may include inputs to allow a user to select whether stimulation will be provided using biphasic (FIG. 2A) or monophasic pulses, and to select whether passive charge recovery will be used, although again these details aren't shown for simplicity.

Stimulation parameter interface 104 may further allow a user to select the active electrodes—i.e., the electrodes that will receive the prescribed pulses. Selection of the active electrodes can occur in conjunction with a leads interface 702, which can include an image 103 of the one or more leads that have been implanted in the patient. Although not shown, the leads interface 102 can include a selection to access a library of relevant images 103 of the types of leads that may be implanted in different patients.

In the example shown in FIG. 7, the leads interface 102 shows an image 103 of a single split-ring lead 33 like that described earlier with respect to FIG. 1B. The leads interface 702 can include a cursor 101 that the user can move (e.g., using a mouse connected to the clinician programmer 70) to select an illustrated electrode 16 (e.g., E1-E8, or the case electrode Ec). Once an electrode has been selected, the stimulation parameter interface 104 can be used to designate the selected electrode as an anode that will source current to the tissue, or as a cathode that will sink current from the tissue. Further, the stimulation parameter interface 104 allows the amount of the total anodic or cathodic current +I or −I that each selected electrode will receive to be specified in terms of a percentage, X. For example, in FIG. 7, the case electrode 12 Ec is specified to receive X=100% of the current I as an anodic current +I. The corresponding cathodic current −I is split between electrodes E5 (0.18*−I), E7 (0.52*−I), E2 (0.08*−I), and E4 (0.22*−I). Thus, two or more electrodes can be chosen to act as anodes or cathodes at a given time using MICC (as described above), allowing the electric field in the tissue to be shaped. The currents so specified at the selected electrodes can be those provided during a first pulse phase (if biphasic pulses are used), or during an only pulse phase (if monophasic pulses are used).

GUI 100 can further include a visualization interface 106 that can allow a user to view an indication of the effects of stimulation, such as electric field image 112 formed on the one or more leads given the selected stimulation parameters. The electric field image 112 is formed by field modelling in the clinician programmer 70. Only one lead is shown in the visualization interface 106 for simplicity, although again a given patient might be implanted with more than one lead. Visualization interface 106 provides an image 111 of the lead(s) which may be three-dimensional.

The visualization interface 106 preferably, but not necessarily, further includes tissue imaging information 114 taken from the patient, represented as three different tissue structures 114a, 114b and 114c in FIG. 7 for the patient in question, which tissue structures may comprise different areas of the brain for example. Such tissue imaging information may comprise a Magnetic Resonance Image (MRI), a Computed Tomography (CT) image or other type of image and is preferably taken prior to implantation of the lead(s) in the patient. Often, one or more images, such as an MRI, CT, and/or a brain atlas are scaled and combined in a single image model. As one skilled in the art will understand, the location of the lead(s) can be precisely referenced to the tissue structures 114i because the lead(s) are implanted using a stereotactic frame (not shown). This allows the clinician programmer 70 on which GUI 100 is rendered to overlay the lead image 111 and the electric field image 112 with the tissue imaging information in the visualization interface 106 so that the position of the electric field 112 relative to the various tissue structures 114i can be visualized. The image of the patient's tissue may also be taken after implantation of the lead(s), or tissue imaging information may comprise a generic image pulled from a library which is not specific to the patient in question.

The various images shown in the visualization interface 106 (i.e., the lead image 111, the electric field image 112, and the tissue structures 114i) can be three-dimensional in nature, and hence may be rendered in the visualization interface 106 in a manner to allow such three-dimensionality to be better appreciated by the user, such as by shading or coloring the images, etc. Additionally, a view adjustment interface 107 may allow the user to move or rotate the images, using cursor 101 for example.

GUI 100 can further include a cross-section interface 108 to allow the various images to be seen in a two-dimensional cross section. Specifically, cross-section interface 108 shows a particular cross section 109 taken perpendicularly to the lead image 111 and through split-ring electrodes E5, E6, and E7. This cross section 109 can also be shown in the visualization interface 106, and the view adjustment interface 107 can include controls to allow the user to specify the plane of the cross section 109 (e.g., in XY, XZ, or YZ planes) and to move its location in the image. Once the location and orientation of the cross section 109 is defined, the cross-section interface 108 can show additional details. For example, the electric field image 112 can show equipotential lines allowing the user to get a sense of the strength and reach of the electric field at different locations. Although GUI 100 includes stimulation definition (702, 104) and imaging (108, 106) in a single screen of the GUI, these aspects can also be separated as part of the GUI 100 and made accessible through various menu selections, etc.

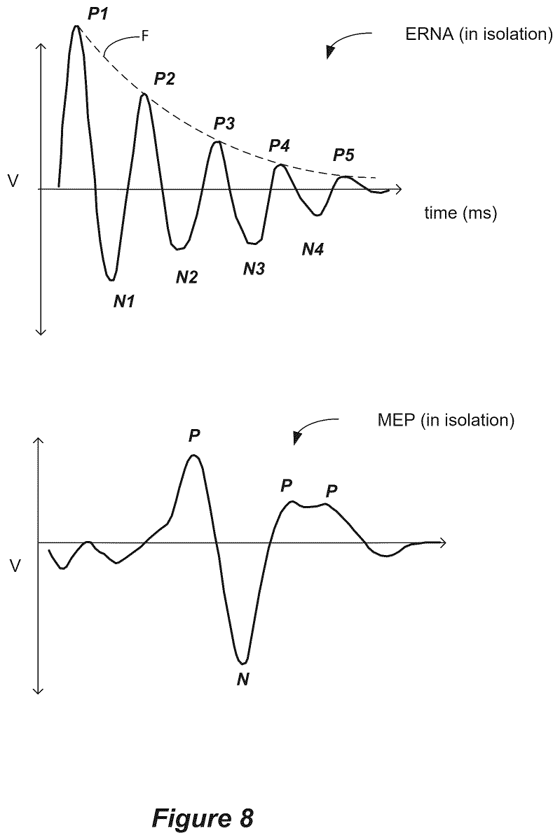

As mentioned above, aspects of this disclosure relate to stimulation systems, and particularly to ETS systems, that include sensing capabilities and thereby allow electrophysical measurements to be made. For example, it has been observed that DBS stimulation in certain positions in the brain can evoke neural responses, i.e., electrical activity from neural elements, which may be measured either from brain itself or from other locations in the body (such as muscles). Such evoked neural responses are referred to herein generally as evoked potentials (EPs). One example of such EPs are resonant neural responses, referred to herein as evoked resonant neural responses (ERNAs). See, e.g., Sinclair, et al., “Subthalamic Nucleus Deep Brain Stimulation Evokes Resonant Neural Activity,” Ann. Neurol. 83(5), 1027-31, 2018. The ERNA responses typically have an oscillation frequency of about 200 to about 500 Hz. Stimulation of the STN, and particularly of the dorsal subregion of the STN, has been observed to evoke strong ERNA responses, whereas stimulation of the posterior subthalamic area (PSA) does not evoke such responses. EPs may be elicited and recorded from additional brain regions, including pallidal and cortical regions, among others. Thus, ERNA may provide a biomarker for electrode location, which can indicate acceptable or optimal lead placement and/or stimulation field placement for achieving the desired therapeutic response. An example of an ERNA in isolation is illustrated in FIG. 8. The ERNA comprises a number of positive peaks Pₙ and negative peaks Nₙ, which may have one or more characteristic amplitudes, lengths, separations, latencies, or other features. The ERNA signal may decay according to a characteristic decay function F. Such characteristics of the ERNA response may provide indications of the brain activity associated with the neural response.

Another example of an EP is motor evoked potentials (MEPs), which are electrical signals recorded from the descending motor pathways or from muscles following stimulation of motor pathways in the brain. An MEP is shown in isolation in FIG. 8 and comprises a number of peaks that are conventionally labeled with P for positive peaks and N for negative peaks. Note that not all MEPs will have the exact shape and number of peaks as illustrated in FIG. 8. According to some embodiments, EPs, such as MEPs, may be used to limit stimulation. Another example of signals that may be indicative of side effects include signals that may indicate internal capsule (IC) activation. Other examples of electrical activity that may be recorded include spontaneous neural activity (local field potentials) as well as other evoked potentials, such as cortical evoked potentials, compound muscle action potentials (CMAPs), evoked compound action potentials (ECAPs), and the like, as well as other electrical properties of the electrode-tissue interface, such as impedance.

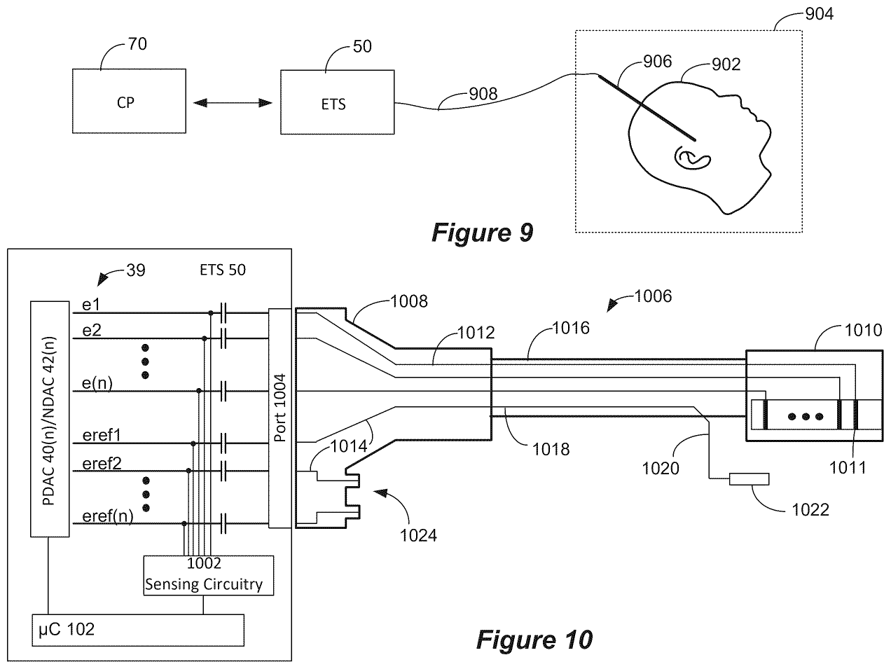

FIG. 9 illustrates an operating room environment wherein in a patient 902 is having one or more electrode leads (e.g., electrode lead 33, FIG. 1B) implanted in their brain. More details of the electrode implantation procedure may be found in U.S. Pat. No. 7,953,497, issued May 31, 2011, the contents of which are incorporated herein by reference. It will be appreciated that significant aspects and equipment (e.g., a stereotactic frame) used during the implantation procedure are omitted from the drawing, for clarity. A sterile field 904 may be established about the patient's head (i.e., the location of the incision). The electrode lead may be inserted into a cannula 906, which is used to advance the lead near to the target location in the patient's brain. As mentioned above, a CP 70 and ETS 50 may be used during the implantation procedure to provide stimulation and/or electrophysical sensing capabilities during the procedure. In the illustrated example, the CP 70 is capable of communicating with the ETS, either via a wired or wireless connection. The ETS is attached to the lead (inside the cannula and not shown) via a cable 908, referred to herein as an OR cable.

The ETS 50 may comprise stimulation circuitry and/or sensing circuitry, as illustrated in FIGS. 3 and 6 and as described above and in the above-incorporated patents/applications. In the ETS environment illustrated in FIG. 9, the OR cable 908 provides electrical connections between each of the electrode nodes 39 (FIGS. 3 and 6) and electrode contacts (e.g., 16, FIGS. 1A, 1B). But consider how the stimulation/sensing circuitry may be implemented in the ETS 50, compared to in an IPG 10. As described above, in an IPG, control of the PDACs 40i and NDACs 42i can allow any of the electrode contacts and/or the case electrode to act as anodes or cathodes. Thus, the circuitry within the IPG can cause one of the electrode contacts to act as an anode and another of the electrode contacts to act as a cathode, thereby providing bipolar stimulation. Alternatively, when using an IPG, the case electrode may serve as an anode or cathode, thereby allowing monopolar stimulation, as illustrated in FIG. 2A. Likewise, the sensing circuitry within an IPG can either sense electrical signals using two (or more) of the electrode contacts of the lead for sensing or it can use one of the lead-based electrode contacts and can use the case as a reference electrode, thereby providing monopolar sensing. But in the context of an ETS 50, an IPG case is not available to serve as a reference electrode. Thus, some external reference may be provided if monopolar stimulation and/or monopolar sensing is to be achieved. Accordingly, aspects of this disclosure relate to methods and systems for providing such reference electrodes in an ETS environment. It should be noted that, while the primary focus of the disclosure is related to providing ways of contacting such reference electrodes that are not located upon the electrode lead(s), the disclosed embodiments may be used to contact other “off-lead” electrodes as well. In other words, the off-lead electrodes may, or may not, be reference electrodes. For example, the off-lead electrodes may be configured to record potentials/currents and/or to pass potentials/currents.

FIG. 10 illustrates an ETS 50, which comprises stimulation circuitry and sensing circuitry, similar to that illustrated in FIG. 6, which is rearranged and abbreviated for clarity. The ETS is configured to communicate with a CP 70, as shown in FIG. 9. The illustrated ETS 50 comprises a microcontroller 102 and DAC circuitry 40/42. The illustrated sensing circuitry 1002 may include the sense amplifier(s) (110, FIG. 6), multiplexer(s) (108, FIG. 6), and ADC(s) (112, FIG. 6), as described above. The illustrated plurality of electrode nodes 39 may be configured to provide stimulation/sensing to lead-based electrodes (e1-en) and for a plurality of reference electrodes that are not lead-based (eref1-erefn). For example, one embodiment of an ETS 50 may comprise 16 electrode nodes for providing stimulation/sensing connections to each of the electrode contacts on two electrode leads (such as electrode leads 18 or 33, FIGS. 1A and 1B, respectively). Such an ETS may also comprise two reference electrode nodes (eref1 and eref2). Each of the electrode nodes may be operatively connected to a port 1004, which may generally be any type of I/O port known in the art. For example, the port 1004 may comprise 18 pins-16 pins connected (or corresponding to) the stimulating/sensing electrode nodes and 2 pins connected (or corresponding to) the reference electrode nodes of the ETS. According to some embodiments, the user can differentially set connections between lead and off-lead electrode. In other words, various of the lead-based electrodes may be shorted to various of the reference (off-lead) electrodes. The actuation may be physical (for example via a toggle or slide on the ETS) and/or may be programmed within the CP, for example.

FIG. 10 also illustrates an embodiment of an OR cable 1006 for connecting the ETS 50 with one or more electrode leads and reference electrodes. The proximal end of the OR cable comprises an ETS connector 1008 configured to fit the port 1004 of the ETS. The distal end of the OR cable comprises one or more lead connectors 1010 configured to connect to one or more electrode leads. In the illustrated embodiment, the lead connector comprises lead connector contacts 1011 that are configured to connect to proximal contacts 21 (FIG. 1A) of an electrode lead. The ETS connector 1008 comprises electrical conductors (e.g., wire, traces, etc.) for the stimulation/sensing electrode nodes 1012 and for the reference electrode nodes 1014. The illustrated OR cable comprises a cable body 1016 connecting the ETS connector 1008 with the lead connector 1010. The cable body 1016 may comprise wires contained within an insulating sheath. The cable body may comprise passive or active grounding and/or shielding.

According to some embodiments, electrical conductor(s) of the ETS connector 1008 may connect one or more of the reference electrode nodes to the cable body, whereby electrical connection to the reference electrode node(s) may be provided to the distal end of the OR cable. For example, one or more wires in the cable body may connect one or more reference nodes to the lead connector (this embodiment is not shown). Alternatively (or additionally), such as in the illustrated embodiment, a wire 1018 within the cable body may connect to a reference electrode node and exit the cable body as a “pig tail” 1020 at the distal end of the cable body. The “pig tail” (i.e., short, single wire) may comprise a connector 1022 for connecting to a reference electrode, as described in more detail below. The connector 1022 may be an alligator clip, a mini-DIN connector, a safety DIN connector, an EEG DIN connector, for example.

According to some embodiments, the ETS connector 1008 may provide access to one or more of the reference electrode nodes at the proximal end of the OR cable. For example, the illustrated embodiment of the ETS connector 1008 comprises jacks 1024 for connecting to reference electrode nodes at the proximal end of the OR cable. It should be appreciated that various embodiments and configurations of an OR cable for providing one or more reference electrode node connections are possible. For example, some embodiments may not include a pigtail connection 1020 at the distal end and may simply include one or more jacks 1024 at the proximal end. Alternatively, some embodiments may include a plurality of pigtail connections at the distal end.

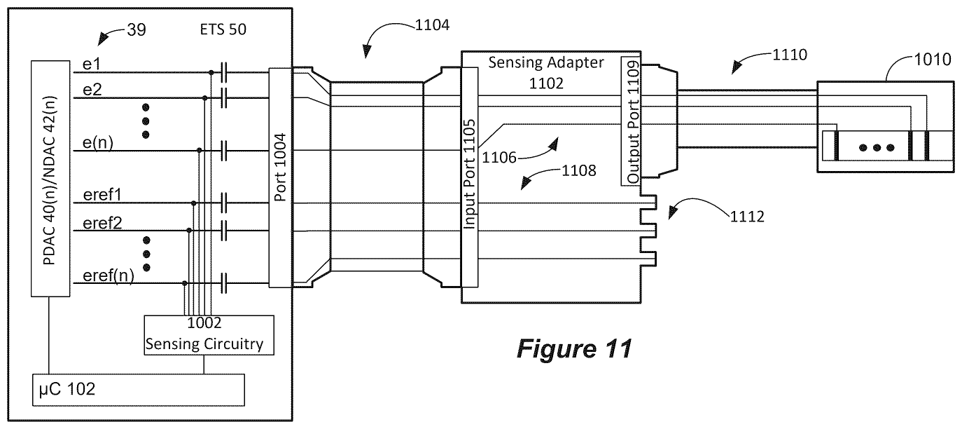

FIG. 11 shows another embodiment for providing reference electrode access to an ETS 50. A sensing adapter 1102 is configured to attach to the port 1004 of the ETS 50 via a connector cable 1104, which may attach to an input port 1105 of the sensing adapter. The sensing adapter 1102 may comprise a first plurality of conductors 1106 corresponding to the stimulation/sensing electrode nodes of the ETS and a second plurality of conductors 1108 corresponding to the reference electrode nodes of the ETS. The first plurality of conductors 1106 may be configured to electrically connect the stimulation/sensing electrode nodes of the ETS to an output port 1109, which may connect to an OR cable 1110, whereby the stimulation/sensing conductors 1106 can electrically communicate with one or more electrode leads attached to one or more lead connectors 1010. The second plurality of conductors 1108 may be configured to electrically connect the reference electrode nodes of the ETS to one or more jack(s)/connector(s) 1112. The jack(s)/connector(s) 1112 allow reference electrode(s) to connect to the sensing adapter using reference electrode cables with mating plugs.

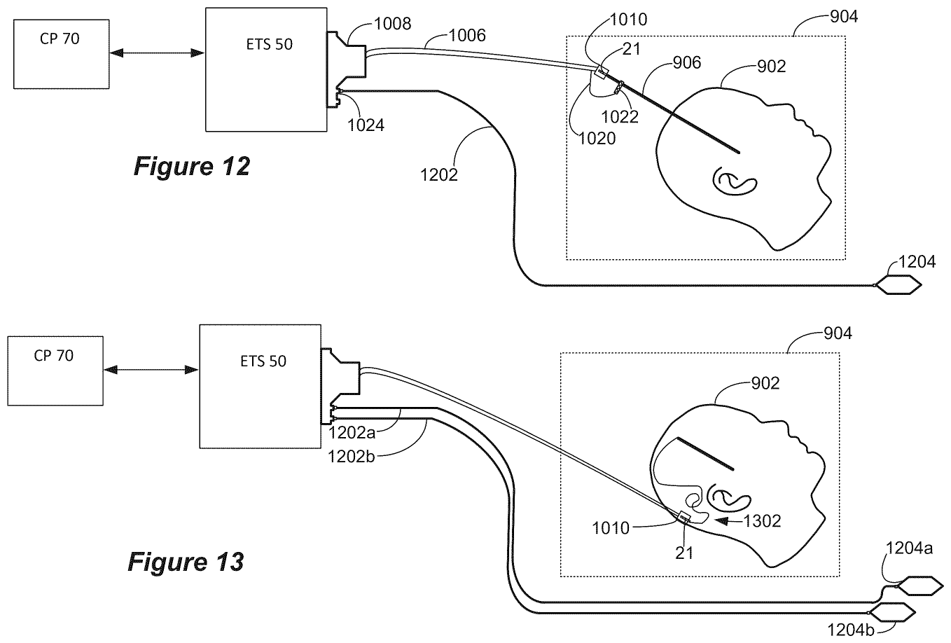

FIG. 12 illustrates an embodiment of an OR cable 1006 (as illustrated in FIG. 10) implemented in an OR environment during the implantation of electrode leads within a patient's brain. The illustrated implementation allows a clinician to provide stimulation and perform electrophysical measurements with respect to reference electrodes as the electrode lead is being placed. As described above, a sterile field 904 is established about the patient's head 902 and a cannula 906 is being used to guide the electrode lead. The ETS connector 1008 of the OR cable is connected to the ETS 50. A lead connector 1010 at the distal end of the OR cable is connected to the proximal contacts 21 of the electrode lead. A connector 1022 on a distal pig tail 1020 is connected to the cannula 906 to provide a reference electrode contact that is located within the sterile field 904 but is remote from the electrode contacts located on the electrode lead. A reference electrode cable 1202 is connected to a jack/connector 1024 of the ETS connector 1008. The reference electrode cable 1202 provides a connection to a second remote reference electrode contact 1204, which may be a patch electrode, for example. The patch electrode may be attached to the patient's torso, arms, legs, etc., and may be inside or outside the sterile field 904.

FIG. 13 illustrates another implementation of modified OR cable 1006 in an OR environment. In this implementation, the electrode lead has been implanted and the proximal portion of the electrode lead 1302 has been tunneled and coiled in a pocket formed behind the patient's ear. The lead connector 1010 of the OR cable is connected to the proximal contacts 21 of the electrode lead. In this illustrated implementation, two reference electrode cables 1202a and 1202b are connected to the jacks 1024 of the ETS connector 1008 and used to provide connections to remote reference electrodes 1204a and 1204b, respectively.

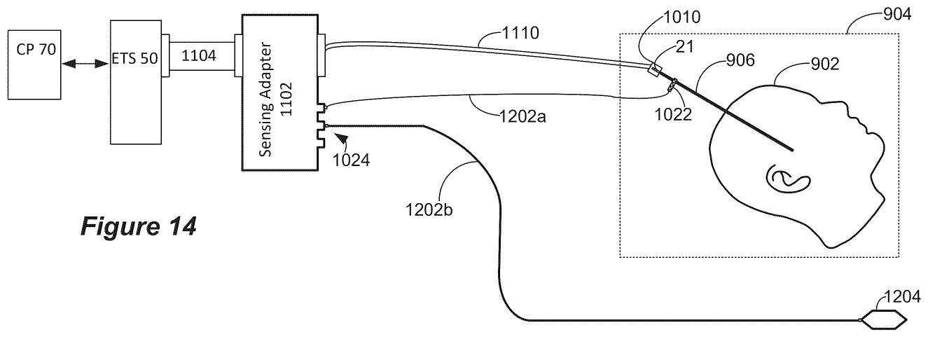

FIG. 14 illustrates an implementation of a sensing adapter 1102 (as illustrated in FIG. 11) implemented in an OR environment during the implantation of electrode leads within a patient's brain. The sensing adapter 1102 is connected to the ETS 50 via a connector cable 1104. An OR cable 1110 is connected to the sensing adapter and a lead connector 1010 at the distal end of the OR cable is connected to the proximal contacts 21 of the electrode lead. In the illustrated embodiment, a first reference electrode cable 1202a is attached to one of the jacks 1024 of the sensing adapter and is used to form a first reference connection to the cannula 906 via the connector 1022 (e.g., an alligator clip). A second reference electrode cable 1202b is connected to a second of the jacks 1024 and is used to form a second reference connection to a reference electrode 1204 (e.g., a patch electrode). A person of skill in the art will appreciate that other configurations using a modified OR cable 1006 and/or a sensing adapter 1102 are possible, based on the description of the embodiments shown in FIGS. 12-14.

Although particular embodiments of the present invention have been shown and described, it should be understood that the above discussion is not intended to limit the present invention to these embodiments. It will be obvious to those skilled in the art that various changes and modifications may be made without departing from the spirit and scope of the present invention. Thus, the present invention is intended to cover alternatives, modifications, and equivalents that may fall within the spirit and scope of the present invention as defined by the claims.

What is claimed is:

1. A system for providing electrical stimulation to a patient's brain using one or more electrode leads implantable in the patient's brain, wherein each of the one or more electrode leads comprise a plurality of distal electrode contacts configured to contact the patient's brain and a plurality of proximal electrode contacts outside the patient's brain, the system comprising:
an operating room (OR) cable comprising:
a proximal end configured to connect to an external trial stimulator (ETS);
a distal end comprising one or more electrode lead connectors configured to connect to the proximal electrode contacts of the one or more electrode leads; and
at least two off-lead electrode connectors configured to connect to off-lead electrode contacts that are not configured within the one or more electrode leads.

2. The system of claim 1, wherein at least one of the off-lead electrode connectors is at the proximal end of the OR cable and at least one of the off-lead electrode connectors is at the distal end of the OR cable.

3. The system of claim 1, wherein the OR cable comprises:
an ETS connector at the proximal end, wherein the ETS connector is configured to connect to the ETS; and
a cable body connected to the ETS connector at its proximal end and connected to the electrode lead connector at its distal end, wherein the cable body comprises a plurality of wires configured within an insulating sheath.

4. The system of claim 3, wherein the ETS comprises:
a plurality of stimulation/sensing electrode nodes configured to provide stimulation waveforms and to sense electrical signals; and
a plurality of reference electrode nodes configured to provide a voltage reference with respect to electrical signals sensed at the stimulation/sensing nodes.

5. The system of claim 4, wherein the ETS connector comprises:
a first plurality of conductors configured to electrically connect to the stimulation/sensing electrode nodes, and
a second plurality of conductors configured to electrically connect to the reference electrode nodes.

6. The system of claim 5, wherein the first plurality of conductors is configured to electrically connect the stimulation/sensing electrode nodes of the ETS to a first one or more of the plurality of wires, wherein the first one or more of the plurality of wires each terminate at a lead connector contact within the one or more lead connectors.

7. The system of claim 5, wherein at least one of the second plurality of conductors is configured to electrically connect one or more of the reference electrode nodes to a second one or more of the plurality of wires, wherein the second one or more of the plurality of wires each terminate at an off-lead electrode connector at the distal end of the OR cable.

8. The system of claim 7, wherein the off-lead electrode connector at the distal end of the OR cable comprises a strand of wire that exits the OR cable body proximal to the one or more lead connectors.

9. The system of claim 8, wherein the strand of wire terminates at an alligator clip, a mini-DIN connector, a safety DIN connector, or an EEG DIN connector.

10. The system of claim 5, wherein at least one of the second plurality of conductors is configured to electrically connect one or more of the reference electrode nodes of the ETS to one or more off-lead electrode connectors configured as part of the ETS connector.

11. The system of claim 10, wherein each of the one or more off-lead electrode connectors configured as part of the ETS connector comprises a jack configured to attach to a proximal end of an off-lead electrode cable that is configured to attach to a off-lead electrode at the distal end of the off-lead electrode cable.

12. The system of claim 11, wherein the off-lead electrode is a patch electrode.

13. The system of claim 10, wherein the ETS connector comprises two or more off-lead electrode connectors.

14. The system of claim 1, further comprising the ETS.

15. The system of claim 1, wherein the off-lead electrode is a reference electrode.

16. A method for providing electrical stimulation to a patient's brain using one or more electrode leads implantable in the patient's brain, wherein each of the one or more electrode leads comprise a plurality of distal electrode contacts configured to contact the patient's brain and a plurality of proximal electrode contacts outside the patient's brain, the method comprising:
providing an operating room (OR) cable comprising:
a proximal end configured to connect to an external trial stimulator (ETS),
a distal end comprising one or more electrode lead connectors configured to connect to the proximal electrode contacts of the one or more electrode leads, and
at least two off-lead electrode connectors configured to connect to off-lead electrode contacts that are not configured within the one or more electrode leads,
connecting the proximal end of the OR cable to the ETS,
connecting the one or more electrode lead connectors to the proximal electrode contacts of the one or more electrode leads,
connecting a first of the at least one of the off-lead electrode connectors to a first off-lead electrode,
configuring the first off-lead electrode to have electrical continuity with the patient, and
using the ETS to provide one or more stimulation waveforms to one or more of the plurality of proximal electrode contacts of the one or more electrode leads.

17. The method of claim 16, wherein the first off-lead electrode is a patch electrode and wherein configuring the first off-lead electrode to have electrical continuity with the patient comprises attaching the patch electrode to the patient's skin.

18. The method of claim 17, wherein attaching the patch electrode to the patient's skin comprises attaching the patch electrode outside a sterile field of the patient.

19. The method of claim 17, wherein attaching the patch electrode to the patient's skin comprises attaching the patch electrode inside a sterile field of the patient.

20. The method of claim 16, wherein the first off-lead electrode is a clip, and wherein configuring the first off-lead electrode to have electrical continuity with the patient comprises attaching the clip to a cannula inserted into the patient's brain.
